(19) United States
(12) Patent Application Publication
(10) Pub. No.: US 2022/0143376 A1
(43) Pub. Date: May 12, 2022

(54) VACCINATION USING HIGH-DENSITY MICROPROJECTION ARRAY PATCH

(71) Applicants: Angus FORSTER, St Lucia, Queensland (AU); Vaxxas Pty Limited, Sydney, New South Wales (AU)

(72) Inventor: Angus FORSTER, Queensland (AU)

(21) Appl. No.: 17/442,558

(22) PCT Filed: Mar. 27, 2020
(86) PCT No.: PCT/AU2020/050296

§ 371 Date: Sep. 23, 2021

Related U.S. Application Data
(60) Provisional application No. 62/919,786, filed on Mar. 29, 2019.

(51) Int. Cl. A61M 37/00; A61K 39/145; C12N 7/00

(52) U.S. Cl. CPC A61M 37/0015; A61K 39/145; A61K 2039/541; A61M 2037/0046; C12N 7/00

(57) ABSTRACT

The present invention relates to microprojection arrays for the delivery of vaccines, in particular the use of polymer high density microprojection arrays for the delivery of vaccines to patients in which the dose of the vaccine delivered may be less than the dose of vaccine delivered by intramuscular injection while providing equal or superior immunogenicity.

### BACKGROUND OF THE INVENTION

The present invention relates to microprojection array patches (MAPs) for the delivery of vaccines, in particular the use of polymer high density microprojection array patches (HD-MAP) for the delivery of vaccines to patients in which the dose of the vaccine delivered is less than the dose of vaccine delivered by intramuscular injection (dose-sparing) while providing equal or superior immunogenicity.

### DESCRIPTION OF THE PRIOR ART

The reference in this specification to any prior publication (or information derived from it), or to any matter which is known, is not, and should not be taken as an acknowledgment or admission or any form of suggestion that the prior publication (or information derived from it) or known matter forms part of the common general knowledge in the field of endeavour to which this specification relates.

Most vaccinations are still delivered intramuscularly using the needle and syringe that was invented in 1853. More efficient vaccine delivery methods are needed to make reduced dose, inexpensive, thermostable, self-administrable, and pain-free vaccinations available, especially in resource-poor countries. Patches that are used to deliver vaccines and drugs into skin have been developed such as the silicon micro-projection skin patch that is dry-coated with vaccine (Fernando et al 2018). Because skin has a high concentration of antigen presenting cells, it is an ideal site to deliver vaccines (Fernando 2010). A study conducted with silicon patches produced an immune response similar to that obtained with the conventional needle and syringe IM vaccination was observed at a single dose of 15 μg.

There are various microprojection/microneedle arrays and methods for administering vaccines via the arrays. One consideration in developing a novel vaccine delivery technology is the ability to manufacture the millions of doses required for vaccinations globally. Cost is a consideration and the array administration must be less expensive or comparable in cost to administration by a needle and syringe. Dissolving needle microarrays have been studied (Rouphael 2017, Hirobe 2015) but it is doubtful whether these dissolving needle patches could be mass produced under aseptic conditions in the quantities necessary and at a cost comparable to the needle and syringe. Another disadvantage of dissolving microneedles is that each clinical vaccination takes a minimum of 20 minutes to 6 hours, depending on the design for the needles, to dissolve in skin. Such a long residence time would slow down mass vaccinations considerably. In contrast, patches made with solid inert synthetic polymers can be mass-produced cheaply. Vaccines are dry-coated onto the polymer with a thin layer of vaccine which dissolves rapidly in skin within 2 minutes after application. Automated coating methods to place vaccines directly onto the array microprojections have been developed such that mass production of vaccine coated patches has been achieved.

When vaccines are delivered to the APCs present in skin using high density microprojection arrays, enhanced immunogenicity and dose reductions compared to the conventional needle and syringe intramuscular injections have been observed in some preclinical model, such as mice. Dose reductions in humans using dry-coated vaccine microprojection array skin patches have not been demonstrated. Studies have shown that dose reductions up to 5-fold compared to IM injection could be achieved in humans by delivering liquid influenza vaccine to skin through a hollow microneedle array (Van Damme Vaccine 2009 27 p 454). Dose reductions would permit greater availability of vaccine doses in situations such as in pandemics where the antigen supply will be limited due to the high demand. In addition, expensive vaccines such as the anti-cervical cancer vaccines could be made more affordable to resource-poor countries if the antigen dose required is reduced.

There is a need for devices and methods of vaccination in which the vaccine is stable and which can be administered at a reduced dose with the same efficacy as a needle and syringe. In addition there is a need for devices for vaccination which can be produced in large quantities under GMP conditions at a low cost which avoid reconstitution and increase the onset of immunogenicity. In addition to dose sparing and the ease of mass manufacture of MAP at a very affordable cost, MAPs have further advantages. MAP dry-coated vaccines are generally more thermostable than liquid vaccines required for injection with the needle and syringe.

### SUMMARY OF THE PRESENT INVENTION

In one broad form, an aspect of the present invention seeks to provide a method of stimulating an immune response in a human, comprising the step of administering to the human a vaccine dose which is coated onto a microprojection array patch (MAP).

In one embodiment the MAP comprises a base and a number of solid, non-porous projections extending from the base made of synthetic polymer, wherein at least one projection comprises an uncoated support section which transitions into end section which is dry-coated with vaccine.

In one embodiment the projections are about 200 to 300 μm in length and about 100 to about 120 μm in width at the base and the density of the projections is from about 1000 to about 5000 projections/cm² and the MAP weighs between 0.1 to 0.6 grams.

In one embodiment the MAP is made of a synthetic polymer.

In one embodiment the synthetic polymer is a liquid crystal polymer.

In one embodiment administration of the composition to the human provides protective immunity against an infection consequent to exposure of the human to a source of antigen.

In one embodiment the human is from 49 to 64 years old.

In one embodiment the human is at least 65 years old.

In one embodiment the dose is at least one dose selected from the group consisting of a 0.5 μg dose, 1 μg dose, 2 μg dose, 2.5 μg dose, 3 μg dose, 4 μg dose, 5 μg dose, 6 μg dose, 7 μg dose, 8 μg dose, 10 μg dose, 15 μg dose, 20 μg dose, 25 μg dose and a 30 μg dose.

In one embodiment the dose is at least one dose selected from the group consisting of a 2.5 μg dose, 5 μg dose, 10 μg dose and a 15 μg dose.

In one embodiment the vaccine dose comprises one or more influenza antigens.

In one embodiment the influenza antigen is a hemagglutinin influenza antigen

In one embodiment the influenza antigen is an influenza A antigen.

In one embodiment the influenza antigen is an influenza B antigen.

In one embodiment the influenza antigen is an influenza C antigen.

In one embodiment the method further includes the step of administering at least one subsequent dose of the vaccine to the human.

In one broad form, an aspect of the present invention seeks to provide a method of stimulating an immune response in a human population, comprising the step of administering to the human population vaccine doses which are dry-coated onto a microprojection array patch (MAP) and inserted into the skin of the humans in the population, wherein the seroconversion rate in the human population is at least 85% as measured at least 8 days after the administration of the vaccine.

In one broad form, an aspect of the present invention seeks to provide a method of stimulating an immune response in a human population, comprising the step of administering to the human population vaccine doses which are dry-coated onto a microprojection array patch (MAP) and inserted into the skin of the humans in the population, wherein the seroprotection rate in the human population is at least 95% as measured at least 8 days after the administration of the vaccine.

In one embodiment the vaccine dose comprises one or more influenza antigens.

In one embodiment the influenza antigen is a hemagglutinin influenza antigen.

In one embodiment the influenza antigen is an influenza A antigen.

In one embodiment the influenza antigen is an influenza B antigen.

In one embodiment the influenza antigen is an influenza C antigen.

In one embodiment the dose comprises between 2.5 to 15 μg hemagglutinin influenza antigen.

In one broad form, an aspect of the present invention seeks to provide a method of stimulating an immune response in a human population, comprising the step of administering to the human population vaccine doses which are dry-coated onto a microprojection array patch (MAP) and inserted into the skin of the humans in the population, wherein the geometric mean titres (GMT) in the human population is at least sixfold greater than the GMT compared to intramuscular injection of the same dose of vaccine as measured at least 8 days after the administration of the vaccine.

In one embodiment the GMT in the human population is from about sixfold to about tenfold greater than the GMT compared to intramuscular injection of the same dose of vaccine as measured at least 8 days after the administration of the vaccine.

In one broad form an aspect of the present invention seeks to provide apparatus for stimulating an immune response in a human, the apparatus comprising a vaccine dose which is coated onto a microprojection array patch (MAP).

In one embodiment the MAP comprises a base and a number of solid, non-porous projections extending from the base made of synthetic polymer, wherein at least one projection comprises an uncoated support section which transitions into end section which is dry-coated with vaccine.

In one embodiment the projections are about 200 to 300 μm in length and about 100 to about 120 μm in width at the base and the density of the projections is from about 1000 to about 5000 projections/cm² and the MAP weighs between 0.1 to 0.6 grams.

In one embodiment the MAP is made of a synthetic polymer.

In one embodiment the synthetic polymer is a liquid crystal polymer.

In one embodiment administration of the composition to the human provides protective immunity against an infection consequent to exposure of the human to a source of antigen.

In one embodiment the human is from 49 to 64 years old.

In one embodiment the human is at least 65 years old.

In one embodiment the dose is at least one dose selected from the group consisting of a 0.5 μg dose, 1 μg dose, 2 μg dose, 2.5 μg dose, 3 μg dose, 4 μg dose, 5 μg dose, 6 μg dose, 7 μg dose, 8 μg dose, 10 μg dose, 15 μg dose, 20 μg dose, 25 μg dose and a 30 μg dose.

In one embodiment the dose is at least one dose selected from the group consisting of a 2.5 μg dose, 5 μg dose, 10 μg dose and a 15 μg dose.

In one embodiment the vaccine dose comprises one or more influenza antigens.

In one embodiment the influenza antigen is a hemagglutinin influenza antigen

In one embodiment the influenza antigen is an influenza A antigen.

In one embodiment the influenza antigen is an influenza B antigen.

In one embodiment the influenza antigen is an influenza C antigen.

In one broad form an aspect of the present invention seeks to provide apparatus for stimulating an immune response in a human population, the apparatus comprising vaccine doses which are dry-coated onto a microprojection array patch (MAP) configured to be inserted into the skin of humans in the population so that the seroconversion rate in the human population is at least 85% as measured at least 8 days after the administration of the vaccine.

In one broad form an aspect of the present invention seeks to provide apparatus for stimulating an immune response in a human population, the apparatus comprising vaccine doses which are dry-coated onto a microprojection array patch (MAP) configured to be inserted into the skin of the humans in the population such that the seroprotection rate in the human population is at least 95% as measured at least 8 days after the administration of the vaccine.

In one embodiment the vaccine dose comprises one or more influenza antigens.

In one embodiment the influenza antigen is a hemagglutinin influenza antigen.

In one embodiment the influenza antigen is an influenza A antigen.

In one embodiment the influenza antigen is an influenza B antigen.

In one embodiment the influenza antigen is an influenza C antigen.

In one embodiment the dose comprises between 2.5 to 15 μg hemagglutinin influenza antigen.

In one broad form an aspect of the present invention seeks to provide apparatus for stimulating an immune response in a human population, the apparatus comprising vaccine doses which are dry-coated onto a microprojection array patch (MAP) configured to be inserted into the skin of the humans in the population such that the GMT in the human population is at least sixfold greater than the GMT compared to intramuscular injection of the same dose of vaccine as measured at least 8 days after the administration of the vaccine.

In one embodiment the GMT in the human population is from about sixfold to about tenfold greater than the GMT compared to intramuscular injection of the same dose of vaccine as measured at least 8 days after the administration of the vaccine.

It will be appreciated that the broad forms of the invention and their respective features can be used in conjunction and/or independently, and reference to separate broad forms is not intended to be limiting. Furthermore, it will be appreciated that features of the method can be performed using the system or apparatus and that features of the system or apparatus can be implemented using the method.

### BRIEF DESCRIPTION OF THE DRAWINGS

Various examples and embodiments of the present invention will now be described with reference to the accompanying drawings, in which: —

FIG. 1A is a photograph of a polymer microprojection array patch.

FIG. 1B is a photograph of the microprojections of the polymer array coated with vaccine using a ink jet coating method.

FIG. 1C is a photograph of a microprojection array applicator.

FIG. 1D is a photograph of the application of the microprojection array to the forearm using the applicator.

FIG. 2 is a scanning electron micrograph of the microprojection array coated with vaccine.

FIGS. 3A and 3B are flow charts of the design for study A and B described in the Examples.

FIG. 4A is a plot of μg of hemagglutinin versus time for the 5 μg dose vaccine.

FIG. 4B is a plot of μg of hemagglutinin versus time for the 15 μg dose vaccine.

FIG. 5 is a plot of hemagglutinin inhibition titer versus several vaccine formulations where the NP designations are microprojection array intradermal administrations and IM is intramuscular injections.

FIG. 6 is a plot of hemagglutinin inhibition titer for day 1 versus day 22 for several vaccine formulations in study A where the NP designations are microprojection array intradermal administrations and IM is intramuscular injections.

FIG. 7 is a plot of hemagglutinin inhibition titer versus time for study A.

FIG. 8 is a plot of microneutralization titer at day 22 for study A.

FIG. 9 is a plot of Hemagglutination inhibition (HAI) titres for subjects in part B at study days 1 (pre-vaccination), 4, 8, 22 and 61. Subjects B were vaccinated with: A/Singapore/GP1908/2015 H1N1 at 15, 10, 5, or 2.5 μg HA/dose delivered by HD-MAPs applied to the volar forearm (MAP-FA-15, MAP-FA-10, MAP-FA-5, MAP-FA-2.5); uncoated HD-MAPs (MAP-FA-0); A/Singapore/GP1908/2015 H1N1 at 15 μg HA/dose delivered by HD-MAP applied to the upper arm (MAP-UA-15); or injected IM as a component of the Afluria® quadrivalent vaccine (IM-QIV-15). Symbols represent the geometric mean titres (GMTs) and the error bars show the 95% confidence intervals.

FIG. 10 is a plot of Microneutralisation titres at day 1 (pre-vaccination) and day 22 for subjects in part B following vaccination with: A/Singapore/GP1908/2015 H1N1 at 15, 10, 5, or 2.5 μg HA/dose delivered by HD-MAPs applied to the volar forearm (MAP-FA-15, MAP-FA-10, MAP-FA-5, MAP-FA-2.5); uncoated HD-MAPs (MAP-FA-0); A/Singapore/GP1908/2015 H1N1 at 15 μg HA/dose delivered by HD-MAP applied to the upper arm (MAP-UA-15); or injected IM as a component of Afluria® quadrivalent vaccine (IM-QIV-15). Columns represent the GMTs, symbols represent the titres from individual subjects and the error bars show the 95% confidence intervals.

FIG. 11A is a plot of the midpoint ELISA titers and FIG. 11B is a plot of the fold change in mid-point titers day 22 vs. day 1 for HA-specific FcR-binding antibodies. Antibodies specific for A/Singapore/GP1908/2015 monovalent purified harvest that engage with dimeric, soluble recombinant FcγRIII were measured by ELISA. Symbols represent individual responses before day 1 and after day 22 immunization where horizontal lines indicate the media response (A); columns with error bars represent the median with interquartile ranges (B).

FIG. 12 is a plot of Influenza-specific IgA titres in saliva samples. Subjects were vaccinated with either: 15 μg of A/Singapore/GP1908/2015 H1N1 delivered by HD-MAP to either the volar forearm (MAP-FA-15) or upper arm (MAP-UA-15), or injected IM as a component of Afluria® quadrivalent vaccine (IM-QIV-15) or uncoated HD-MAPs (MAP-FA-0). Four time-points were measured: pre-vaccination (Day 1), day 4, 8 and 22. The absorbance values per group for each time-point were averaged and compared against day 1, and the fold-change compared with pre-vaccination (day 1) plotted. Symbols represent the means from all subjects per group and the error bars show the 95% confidence intervals.

FIG. 13A to 13F are plots of memory cell (MBC) frequencies pre- and post-vaccination. The frequencies of HA-specific MBC were assessed in cryopreserved PMBC samples by flow cytometry. Samples were gated for live, CD19+, IgD-B cells and specificity determined based upon binding to A/Michigan/2015 probes alone or in combination with A/New Caledonia/1999 or a stabilized H1N1 stem probe. FIGS. 13A and 13B are A/Michigan/2015 H1N1; FIGS. 13C and 13D are A/New Caledonia/1999; FIGS. 13E and 13F are H1 stem. Results are expressed as a frequency of probe-binding cells at day 1 and day 22 in FIGS. 13A, 13C and 13E with symbols representing individual responses before day 1 and after day 22 immunization, and horizontal lines indicating the median response, whilst fold-change at day 22 compared with baseline is shown in FIGS. 13B, 13D and 13F, with columns representing the median fold-change and error bars representing the median with interquartile ranges.

### DETAILED DESCRIPTION OF THE PREFERRED EMBODIMENTS

In one broad form, an aspect of the present invention relates to microprojection arrays for the delivery of vaccines, in particular the use of polymer high density microprojection arrays for the delivery of vaccines to patients in which the dose of the vaccine delivered is less than the dose of vaccine delivered by intramuscular injection (dose-sparing) while providing equal or superior immunogenicity. The devices and methods of the present invention also provide thermostability of the vaccine, ease of use, acceptability and the avoidance of reconstitution of vaccines.

Influenza causes significant morbidity and mortality in adults over 65 years of age and strategies to improve vaccine coverage, immunogenicity and effectiveness in this age group are required. Currently IIVs for this population group require chemical adjuvants such as MF59 or high doses of antigen (such as 60 μg HA per strain per dose) to achieve satisfactory immune response. The enhanced immunogenicity seen in MAP delivery indicates that HD-MAPs provide an alternative approach to increased vaccine dosages.

Furthermore, the exceptional thermostability of the vaccine on the MAP compared with standard formulations would eliminate dependence on the cold-chain and reduce vaccine wastage due to could-chain excursions. A more stable vaccine would also remove the need to overload the patch to compensate for lost potency during the shelf-life of the vaccine. Use of the devices and the methods of the present invention could increase the number of vaccine doses that can be produced from the primary vaccine manufacturing facility in a season, or in a pandemic as the amount of antigen required per dose would be reduced. Global capacity for seasonal influenza production declined between 2013 and 2015 due to the switch from TIV to QIV formulations and pandemic vaccine production is dependent on the implementation of dose-sparing strategies. Dose-sparing and rapid onset of protective immunity would also be a valuable attribute for many vaccines of global health importance such as inactivated poliovirus vaccine or yellow fever vaccine where use is limited by chronic supply constraints or by cost. These vaccines are often needed in low-resource settings where other key attributes of the present invention such as thermostability, ease of use, acceptability and the avoidance of reconstitution would also be beneficial.

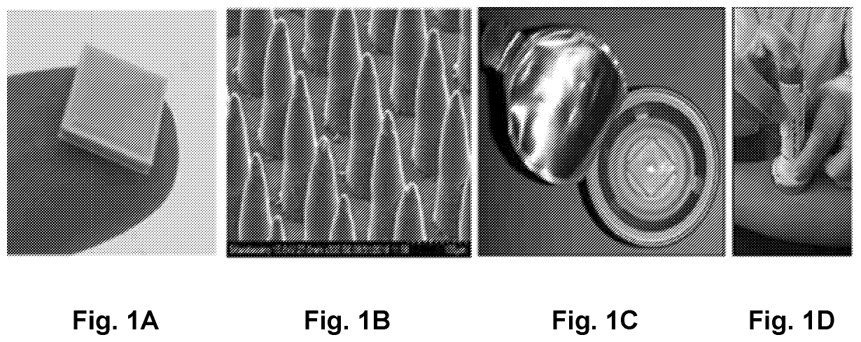

The devices and methods of the present invention include a microprojection array patch (MAP) in which the patch has a width W and a breadth B with the projections being separated by spacing. The projections may be provided in an array that is defined by a regular iteration of microprojections along a square or rectangular arrangement, but other arrangements of projections such as circular arrangement of the projections that are compatible with rotational spray coating may also be used. In order to further improve or enhance the targeting accuracy, the substrate may be designed such that the features to be coated are located on radial lines from the center point of the rotation or located on concentric circles or on a continuous spiral. The substrate may be designed such that the feature spacing on each arc is designed to match an integer number of steps of the motor for a given radius. Each projection includes a tip for penetrating tissue of the biological subject and projections will typically have a profile which tapers from the base to the tip (FIGS. 1A to 1D).

The microprojection arrays may be divided into areas such that a different vaccine antigen or other substance such as an excipient may be coated in each area. For example, the microprojection array may be divided in half or into four equal quadrants where different vaccine antigens or other substances such as excipients may be applied. These areas may have equal numbers of microprojections or unequal numbers of microprojections. In other embodiments some of the microprojections may be uncoated.

The microprojection arrays may have a density of projections of between 1,000 to 7500 per cm², or from 1500 to 7500 per cm² or from 1500 to 5000 per cm² or from 1500 to 2500 per cm² or from 2000 to 7500 per cm² or from 2000 to 5000 per cm² or from 2000 to 4000 per cm² or from 2000 to 3000 per cm² or from 2500 to 7500 per cm² or from 2500 to 5000 per cm² or from 2500 to 4000 per cm² or from 2500 to 3000 per cm² or from 3000 to 7500 per cm², or from 3000 to 5000 per cm² or from 3000 to 4000 per cm² or from 4000 to 7500 per cm² or from 4000 to 5000 per cm² or from 5000 to 7500 per cm². The applicators of the present invention are often utilized to project high density microprojection arrays into the skin. Such high-density arrays are microprojection arrays of sufficient size and density such that forces that can be applied manually will be insufficient to overcome the elasticity of the skin. The projections are typically separated by between 10 μm and 200 μm, between 30 μm and 150 μm, between 50 μm and 120 μm and more typically between 70 μm and 100 μm, leading to patches having between 10 and 1000 projections per mm² and more typically between 1000 and 3000 projections per mm².

The length of the projections may be from 100 μm to 700 μm or from 100 μm to 600 μm or from 100 μm to 500 μm or from 100 μm to 400 μm or from 100 μm to 300 μm or from 100 μm to 250 μm or from 100 μm to 200 μm or from 150 μm to 700 μm or from 150 μm to 600 μm or from 150 μm to 500 μm or from 150 μm to 400 μm or from 150 μm to 300 μm or from 150 μm to 250 μm or from 150 μm to 200 μm or from 200 μm to 700 μm or from 200 μm to 600 μm or from 200 μm to 500 μm or from 200 μm to 400 μm or from 200 μm to 300 μm or from 200 μm to 250 μm or from 225 μm to 700 μm or from 225 μm to 600 μm or from 225 μm to 500 μm or from 225 μm to 400 μm or from 225 μm to 300 μm or from 225 μm to 250 μm or from 250 μm to 700 μm or from 250 μm to 600 μm or from 250 μm to 500 μm or from 250 μm to 400 μm or from 250 μm to 300 μm.

The projections may have one or more step shoulders (discontinuities). In the event that a discontinuities are provided, this is typically located so that at least one discontinuity reaches the dermis, penetration of the projection stops, with the tip extending into the dermal layer. Typically the discontinuity is located from the end of the tip at between 50 and 200 μm, between 50 and 190 μm, between 50 and 180 μm, between 50 and 170 μm, between 50 and 160 μm, between 50 and 150 μm, between 50 and 140 μm, between 50 and 130 μm, between 50 and 120 μm, between 50 and 110 μm, between 50 and 100 μm, between 50 and 90 μm, between 50 and 80 μm, 60 and 200 μm, between 60 and 190 μm, between 60 and 180 μm, between 60 and 170 μm, between 60 and 160 μm, between 60 and 150 μm, between 60 and 140 μm, between 60 and 130 μm, between 60 and 120 μm, between 60 and 110 μm, between 60 and 100 μm, between 60 and 90 μm, between 60 and 80 μm, 70 and 200 μm, between 70 and 190 μm, between 70 and 180 μm, between 70 and 170 μm, between 70 and 160 μm, between 70 and 150 μm, between 70 and 140 μm, between 70 and 130 μm, between 70 and 120 μm, between 70 and 110 μm, between 70 and 100 μm, between 70 and 90 μm, between 70 and 80 μm, between 80 and 200 μm, between 80 and 190 μm, between 80 and 180 μm, between 80 and 170 μm, between 80 and 160 μm, between 80 and 150 μm, between 80 and 140 μm, between 80 and 130 μm, between 80 and 120 μm, between 80 and 110 μm, between 80 and 100 μm, between 80 and 90 μm, between 90 and 200 μm, between 90 and 190 μm, between 90 and 180 μm, between 90 and 170 μm, between 90 and 160 μm, between 90 and 150 μm, between 90 and 140 μm, between 90 and 130 μm, between 90 and 120 μm, between 90 and 110 μm, between 90 and 100 μm, between 100 and 200 μm, between 100 and 190 μm, between 100 and 180 μm, between 100 and 170 μm, between 100 and 160 μm, between 100 and 150 μm, between 100 and 140 μm, between 100 and 130 μm between 100 and 120 μm, between 100 and 110 μm. The discontinuity may provide for greater loading of the drug/vaccine/excipient onto the microprojection.

The microprojection array may be made of any suitable materials including but not limited to metals, silicon, polymers, and plastic. Polymers and plastics are preferred materials including but not limited to liquid crystal polymers. The overall mass of some embodiments of the microprojection array is about 0.3 gm. The microprojection array may have an overall weakly convex shape of the patch to improve the mechanical engagement with skin and mitigate the effect of high-speed rippling application: a ‘high velocity/low mass’ system. The microprojection array may have a mass of less than 1 gram, or less than 0.9 grams or less than 0.8 grams or less than 0.7 grams, or less than 0.6 grams or less than 0.5 grams or less than 0.6 grams, or less than 0.5 grams or less than 0.4 grams or less than 0.3 grams or less than 0.2 grams or less than 0.1 grams or less than 0.05 grams. The microprojection array may have a mass of about 0.05 grams to about 2 grams, or from about 0.05 grams to about 1.5 grams or from about 0.05 grams to about 1.0 grams or from about 0.05 grams to about 0.9 grams, or from about 0.05 grams to about 0.8 grams or from about 0.05 grams to about 0.7 grams, or from about 0.05 grams to about 0.6 grams or from about 0.05 grams to about 0.5 grams or from about 0.05 grams to about 0.4 grams, or from about 0.05 grams to about 0.3 grams or from about 0.05 grams to about 0.2 grams, or from about 0.05 grams to about 0.1 grams or from about 0.1 grams to about 1.0 grams or from about 0.1 grams to about 0.9 grams, or from about 0.1 grams to about 0.8 grams or from about 0.1 grams to about 0.7 grams, or from about 0.1 grams to about 0.6 grams or from about 0.1 grams to about 0.5 grams or from about 0.1 grams to about 0.4 grams, or from about 0.1 grams to about 0.3 grams or from about 0.1 grams to about 0.2 grams. In one embodiment of the applicator/microprojection system the mass of the array is about 0.3 grams, the array is projected at a velocity of about 20-26 m/s by the applicator.

In some embodiments, more than one coating may be applied to the same projection. For instance, different coatings may be applied in one or more layers to provide the same or different materials for delivery to the tissues within the subject at the same time or different times if the layers dissolve in sequence.

The amount of antigen used in the devices and methods of the present invention include amounts necessary to provide an immune response. The dose may be 0.1 μg dose, 0.5 μg dose, dose, 2 μg dose, 2.5 μg does, 3 μg dose, 4 μg dose, 5 μg dose, 6 μg dose, 7 μg dose, 8 μg dose, 9 μg dose, 10 μg dose, 15 μg dose, 20 μg dose, 25 μg dose and a 30 μg dose. The dose may range between about 1 μg to about 100 m, from about 1 μg to about 75 μg, from about 1 μg to about 50 μg, from about 1 μg dose to about 25 μg, from about 1 μg to about 15 μg, from about 1 μg to about 10 μg, from about 1 μg to about 5 μg, from about 2.5 μg to about 100 m, from about 2.5 μg to about 75 μg, from about 2.5 μg to about 50 μg, from about 2.5 μg dose to about 25 μg, from about 2 μg to about 15 μg, from about 2.5 μg to about 10 μg, from about 2.5 μg to about 5 μg, from about 5 μg to about 100 m, from about 5 μg to about 75 μg, from about 5 μg to about 50 μg, from about 5 μg dose to about 25 μg, from about 5 μg to about 15 μg, from about 5 μg to about 10 μg, from about 10 μg to about 100 m, from about 10 μg to about 75 μg, from about 10 μg to about 50 μg, from about 10 μg dose to about 25 μg, from about 10 μg to about 15 μg, from about 15 μg to about 100 m, from about 15 μg to about 75 μg, from about 15 μg to about 50 μg, from about 15 μg dose to about 25 μg, from about 20 μg to about 100 m, from about 20 μg to about 75 μg, from about 20 μg to about 50 μg, from about 20 μg dose to about 25 μg, Each dose may include multiple antigens or multiple substances.

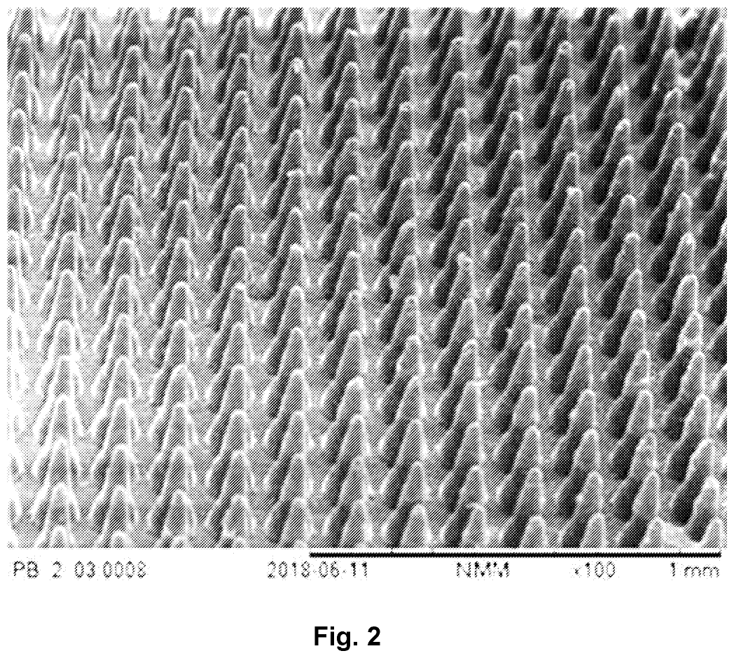

In preferred embodiments the microprojections of the microprojection array are coated by an aseptic print-head type device which rapidly provides small droplets which dry quickly on the microprojections. In preferred embodiments the coating such as a vaccine formulation rapidly dries on the top portion of the microprojection to increase the amount of vaccine (FIG. 2) that can be delivered. The aseptic print head device may deliver multiple drops to the microprojections either sequentially or in an alternating fashion.

The devices and methods of the present invention provide equivalent or superior antibody titer using MAP vaccine delivery to skin compared to the conventional needle and syringe IM injection when using a lesser dose of vaccine. Thus, the devices and methods of the present invention provide for multiple-fold dose reductions using MAP vaccine delivery to skin compared to the conventional needle and syringe IM injection. The device and methods of the present invention provide for between 1.1 fold to 100 fold or from 1.1 fold to 50 fold or from 1.1 fold to 25 fold or from 1.1 fold to 20 fold or from 1.1 to 15 fold or from 1.1 to 10 fold or from 1.1 to 5 fold or from 1.5 fold to 100 fold or from 1.5 fold to 50 fold or from 1.5 fold to 25 fold or from 1.5 fold to 20 fold or from 1.5 to 15 fold or from 1.5 to 10 fold or from 1.5 to 5 fold or from 2 fold to 100 fold or from 2 fold to 50 fold or from 2 fold to 25 fold or from 2 fold to 20 fold or from 2 to 15 fold or from 2 to 10 fold or from 2 fold to 5 fold or from 3 fold to 100 fold or from 3 fold to 50 fold or from 3 fold to 25 fold or from 3 fold to 20 fold or from 3 to 15 fold or from 3 to 10 fold or from 3 to 5 fold or from 4 fold to 100 fold or from 4 fold to 50 fold or from 4 fold to 25 fold or from 4 fold to 20 fold or from 4 to 15 fold or from 4 to 10 fold or from 4 to 5 fold or from 5 fold to 100 fold or from 5 fold to 50 fold or from 5 fold to 25 fold or from 5 fold to 20 fold or from 5 to 15 fold or from 5 fold to 10 fold reduction of vaccine as compared to IM injection.

Other advantages of MAP vaccine delivery over the needle and syringe are; because the vaccine is dry coated onto MAP it is more thermostable, and also do not leave hazardous waste. Influenza vaccine was stable for at least 12 months when stored up to temperature of 40° C. dry-coated on the HD-MAP. This is especially helpful in resource-poor countries where it is difficult to maintain the cold chain. Another advantage is that the MAP micro-projections are invisible to the naked eye, and therefore will be invaluable in vaccinating people and children who have the fear of needles. The higher density of micro-projections in the HD-MAPS induce higher danger signals in the skin during vaccination which possibly lead to physical adjuvantation and enhance immunogenicity.

The devices and methods of the present invention provide higher geometric mean titres (GMTs) of antibodies using MAP vaccine delivery to skin compared to the conventional needle and syringe IM injection measured at the same time point. The device and methods of the present invention provide for between 2 fold to 500 fold or from 2 fold to 100 fold or from 2 fold to 50 fold or from 2 fold to 25 fold or from 2 fold to 20 fold or from 2 to 15 fold or from 2 to 10 fold or from 2 to 5 fold or from 5 fold to 500 fold or from 5 fold to 100 fold or from 5 fold to 50 fold or from 5 fold to 25 fold or from 5 fold to 20 fold or from 5 to 15 fold or from 5 to 10 fold or from 10 fold to 500 fold or from 10 fold to 100 fold or from 10 fold to 50 fold or from 10 fold to 25 fold or from 10 fold to 20 fold or from 10 to 15 fold or from 20 fold to 500 fold or from 20 fold to 100 fold or from 20 fold to 50 fold or from 20 fold to 25 fold or from 50 to 500 fold or from 50 fold to 100 fold increase in GMT as compared to IM injection.

The detection of antibodies post-vaccination occurs more quickly using MAP vaccine delivery to skin compared to the conventional needle and syringe IM injection.

The devices and methods of the present invention provide a protective immune response in a population employing relatively low doses of antigens to infectious agents (e.g. influenza).

Seroresponsive means an increase in HAI antibody titer of at least fourfold with a minimum post vaccination titer of 40. Seroprotection means achievement of minimum post vaccination HAI titer of 40 among subjects with prevaccination titers of <40. Seroconversion rate for anti-HA antibody response is defined as the proportion of subjects in each group having protective post-vaccination titer ≥1:40. The seroprotection rate is the percentage of subjects who have an HAI titer before vaccination of <1:10 and ≥1:40 after vaccination. However, if the initial titer is ≥1:10 then there needs to be at least a fourfold increase in the amount of antibody after vaccination.

In another aspect of the invention it is provided for a composition, method or use as claimed herein wherein the immune response produced by administration of the compositions of the present invention induces functional (HAI) antibodies in the majority of elderly recipients in a dose dependent fashion. In certain embodiments the composition will induce a neutralizing antibody response of greater than a titer about 50 after 7 days or after 14 days or after 28 days. In other embodiments the composition will induce a neutralizing antibody response of greater than about a titer of 100 after 7 days or after 14 days or after 28 days. In other embodiments the composition will induce a neutralizing antibody response of greater than a titer of about 150 after 7 days or after 14 days or after 28 days. In other embodiments the composition will induce a neutralizing antibody response of greater than about 200 after 7 days or after 14 days or after 28 days.

Accordingly, in one aspect of the invention it is provided for a composition, method or use as claimed herein wherein the immune response produced by administration of the composition in a population meets or exceeds one of the following criteria:
a seroconversion rate of greater than 50%, greater than 55%, greater than 60%, greater than 65%, greater than 70%, greater than 75% or greater than 85%, greater than 90%, greater than 95%, or greater than 99%.
a seroprotection rate of greater than 50%, greater than 55%, greater than 60%, greater than 65%, greater than 70%, greater than 75% or greater than 85%, greater than 90%, greater than 95%, or greater than 99%.
an average of two-fold, three-fold, four-fold, five-fold, six-fold, seven-fold, eight-fold, nine-fold, ten-fold or greater increase in neutralizing antibody titer at post vaccination.

An “effective amount” when referring to the amount of a vaccine composition administered to the human, refers to that amount or dose of the composition that, when administered to the subject is an amount sufficient for therapeutic efficacy (e.g., an amount sufficient to stimulate an immune response in a subject, an amount sufficient to provide protective immunity in the subject).

The vaccine compositions can be administered alone or as admixtures with conventional excipients, for example, pharmaceutically, or physiologically, acceptable organic, or inorganic carrier substances which do not deleteriously react with the vaccine composition. Substances that stabilize the vaccine composition may be used in the vaccine composition. While conventional vaccines may contain adjuvants to boost the immune response the formulations of the present invention are preferably used without adjuvants.

The dosage and frequency (single or multiple doses) administered to a subject can vary depending upon a variety of factors, including, for example, prior exposure to an infection consequent to exposure to the antigen: health, body weight, body mass index, and diet of the subject or health-related problems. Other therapeutic regimens or agents can be used in conjunction with the methods and compositions, proteins or polypeptides of the present invention.

The composition can be administered to the human in a single dose or in multiple doses, such as at least two doses. When multiple doses are administered to the subject, a second or third dose can be administered days (e.g., 1, 2, 3, 4, 5, 6, 7), weeks (e.g., 1, 2, 3, 4, 5, 6, 7, 8, 9, 10), months (e.g., 1, 2, 3, 4, 5, 6, 7, 8, 9, 10) or years (e.g., 1, 2, 3, 4, 5, 6, 7, 8, 9, 10) after the initial dose. For example, a second dose of the composition can be administered about 7 days, about 14 days or about 28 days following administration of a first dose of the composition that includes the fusion protein.

Ranges may be expressed herein as from about one particular value, and/or to about another particular value. When such a range is expressed, another aspect includes from the one particular value and/or to the other particular value. Similarly, when values are expressed as approximations, by use of the antecedent about, it will be understood that the particular value forms another aspect. It will be further understood that the endpoints of each of the ranges are significant both in relation to the other endpoint, and independently of the other endpoint.

### EXAMPLES

### Example 1

### Trial Subjects and Study Design

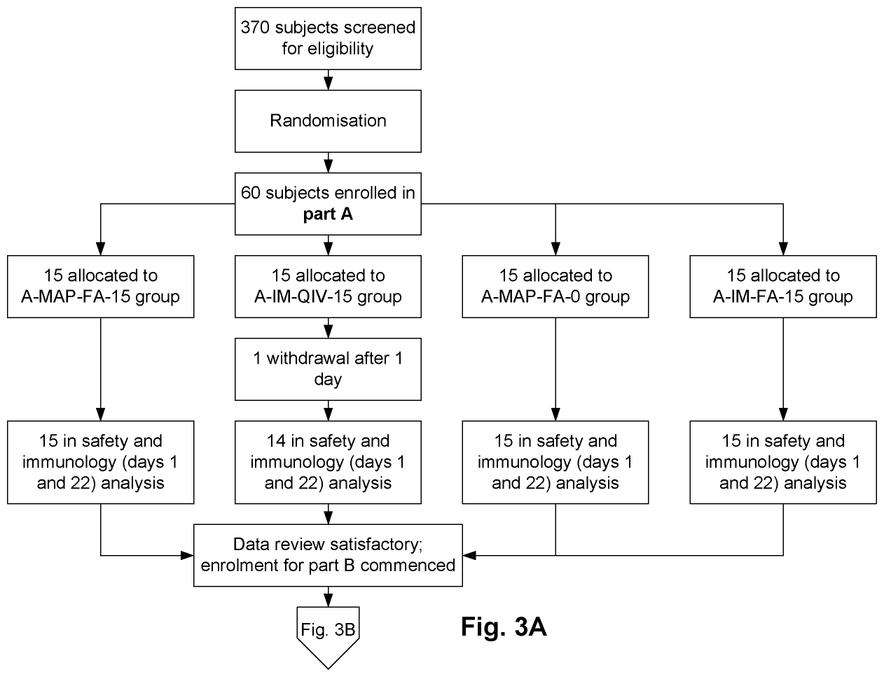

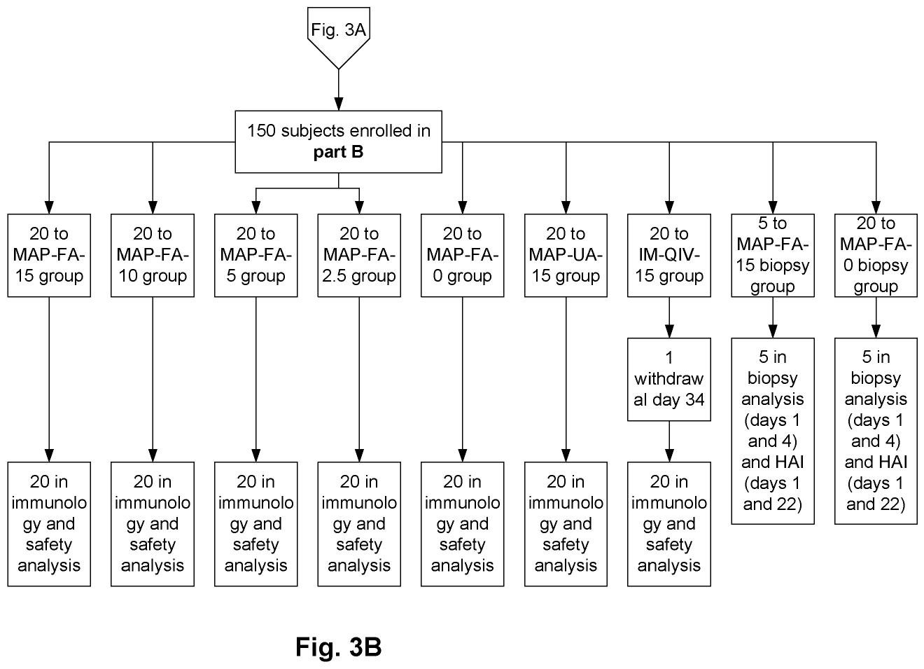

Two hundred and ten (210) healthy subjects total (age 18 to 50 years, BMI of 18 to 30 kg/m²), of whom at least 30% are male, and at least 30% are female, were recruited for the study. Part A: Sixty (60) healthy subjects (4 groups of 15); Part B: One hundred and fifty (150) healthy subjects (7 groups of 20, plus 2 groups of 5 with skin punch biopsies performed). The study design is shown in the FIGS. 3A and 3B. In addition to these groups, further 10 subjects were recruited to do biopsies of the vaccinated sites in the part B of the study.

The Examples described below were performed in two parts. The study was a two-part, randomized, partially double-blind, placebo-controlled trial conducted at Nucleus Network Pty Ltd (Melbourne, VIC). The primary objective was to measure the safety and tolerability of A/Singapore/GP1908/2015 H1N1 (A/Sing) monovalent vaccine delivered by HD-MAP in comparison to an uncoated HD-MAP and IM injection of a quadrivalent seasonal influenza vaccine (QIV) delivering approximately the same dose of A/Sing HA protein. Exploratory outcomes were: to evaluate the immune responses to HD-MAP application to the forearm with A/Sing at 4 dose levels in comparison to IM administration of A/Sing at the standard 15 μg HA per dose per strain, and to assess further measures of immune response through additional assays and assessment of the local skin response via punch biopsy of the HD-MAP application sites.

In all experiments, the subjects were vaccinated once, their blood was collected before vaccination (day 1) and after vaccination (days 4, 8, 22 and 61)) for analyses. The first part (A) consisted of 4 groups of 15 subjects each were vaccinated with one of the following: 1) MAP control without any antigen (A-MAP-FA-0) applied to volar forearm; 2) MAP A/Singapore/GP1908/2015 [H1N1], 15 μg haemagluttinin [HA] per dose), applied to the forearm (A-MAP-FA-15); 3). 15 μg of the same H1N1 HA antigen injected intramuscularly (IM) into the deltoid muscle (A-IM-ASing15); 4). Afluria® Quadrivalent 2017/18 (Segirus™, USA) containing 15 μg of the same H1N1 HA antigen, with an additional 3 strains of influenza antigen injected intramuscularly (IM) into the deltoid muscle (A-IM-QIV-15). In the first part of the experiment, antibody responses at days 1, 22 were measured by ELISA and hemagglutination inhibition assay (HAI).

The second part (B) of the experiment was performed after the evaluation of the part (A) study results. The second part of the study consisted of 7 groups of 20 subjects each. Five groups were vaccinated with A/Singapore/GP1908/2015 (H1N1) using the MAP applied to the forearm at decreasing doses (15, 10, 5, 2.5, and 0 μg HA) (MAP-FA-15, MAP-FA-10, MAP-FA-5, MAP-FA-2.5, and MAPFA-0, respectively). An additional group was vaccinated with 15 μg HA in the upper arm overlying the deltoid muscle using the MAP (MAP-UA-15). The final group was vaccinated with Afluria® Quad 2018 (Segirus™, Australia) containing 15 μg of the same H1N1 HA antigen injected intramuscularly (IM) into the deltoid (IM-QIV-15). The antibody responses at day 1(pre), 4, 8 22, and 61 post vaccinations were measured using hemagglutination inhibition Assays (HAI) and virus microneutralisation assays (VMN) (days 1 and 22 only). Saliva also collected at days 1, 4, 8, and 22 post vaccination. The saliva samples were analysed using ELISA to determine influenza antigen specific secretory IgA.

Abbreviations: A/Sing=Haemagluttinin of split inactivated A/Singapore/GP1908/2015 (H1N1) virus. IM 15 ug Quad=Afluria® Quad 2018 (Segirus™, Australia) vaccine containing 15 μg of A/Sing HA antigen injected intramuscularly (IM) into the deltoid. SD=standard deviation; N=North, E=East, S=South, W=West

### Example 2

### Micro Projection Array Patch (MAP)

The MAPs used in this study are 10×10 mm square containing approximately 3136 micro-projections with a density of 5,000/cm². Each micro-projection is approximately 250 μm in length, 120 μm in width at the base and tapers to a sharp point of less than 25 μm (FIG. 2). Vaccine was aseptically applied to 163 the tips of gamma-irradiated (≥25 kGy, Steritech, Australia) HD-MAPs using a ‘Direct-jet’ process 164 (Vaxxas Pty Ltd, Australia) that deposits individual droplets onto the tip of each projection.

HD-MAPs were produced to deliver two different doses of A/Sing, 2.5 μs and 5.0 μs (referred to as 2.5 μg and 5 μg HD-MAPs), as well as uncoated (placebo) HD-MAPs. Following coating, HD-MAPs were placed into aluminium MediCan containers (Amcor, UK), foil-sealed, and stored at 2-8° C. with desiccant until use. The antigen-coated HD-MAPs were used within 6 months of manufacture.

The MAP was applied to the skin with a hand-held spring device applicator that ejected the patch at a velocity of 20 m/s±2 m/s, and the micro-projections penetrated the epidermis and dermis to an average depth of around 125 μm.

The synthetic polymer MAPs were produced by injection moulding. The choice of the polymer material was based on the ability of the polymer to form the micro-projections, the hardness of the polymer to enable effective skin penetration, appropriate polymer ductility, material compatibility with gamma irradiation sterilisation, and the biocompatibility of the synthetic polymer when in contact with the skin tissues.

### Example 3

### Vaccines

cGMP inactivated split influenza A/Singapore/GP1908/2015 (H1N1), (IVR-180A) virus vaccine was obtained from Seqirus Pty Ltd, Australia (Monovalent Pooled Harvest (MPH). To ensure the haemagluttinin (HA) antigen retains potency during MAP coating and on subsequent storage, a stabilising excipient Sulphobutyl Ether Beta Cyclodextrin (SBECD, Captisol®, Cydex Pharmaceuticals, Kansas, USA)) was added to vaccine solution used with MAPs. 4.8 mg/ml HA was mixed with a small volume of 40% w/v SBECD solution (in water for irrigation, Baxter) to reach a 2% w/v of SBECD solution with A/Singapore (coating solution). For monovalent A/Singapore IM injection, the antigen preparation was diluted with sterile pH tested saline. For the quadrivalent vaccine IM injections, commercially available vaccine was used. In the first part of the study, Afluria® Quadrivalent, Influenza vaccine 2017-2018 (Northern Hemisphere) from Segirus™, USA, was used. For Part B, Afluria® Quad vaccine 2018 (Southern Hemisphere) was used. Afluria® Quadrivalent Northern Hemisphere 2017/18 vaccine contained a nominal 15 μg of HA of each of the following split inactivated virus types: A/Singapore/GP1908/2015 (H1N1), IVR-180A; A/Hong Kong/4801/2014 (H3N2), NYMC X-263B; B/Phuket/3073/2013 BVR-1B; and B/Brisbane/46/2015. Afluria® Quad Southern Hemisphere 2018 vaccine contained a nominal 15 μg HA of each of the following virus types: A/Michigan/45/2015 (H1N1) pdm09—like virus (A/Singapore/GP1908/2015 (IVR-180A)); A/Singapore/INFIMH-16-0019/2016 (H3N2) —like virus (A/Singapore/INFIMH-16-0019/2016 (IVR-186)); B/Phuket/3073/2013—like virus (B/Phuket/3073/2013 (BVR-1B)); and B/Brisbane/60/2008—like virus (B/Brisbane/46/2015).

To produce the A/Sing MAPs, the sterile A/Singapore antigen was added to the filter sterilised SBECD solution and coated onto the tips of MAP micro-projections by a direct jet coating method. The A/Singapore antigen was received as a suspension of particles in phosphate buffered saline and therefore the final solid formulation of antigen on the MAP consists of HA protein, other proteins (from the A/Singapore bulk), SBECD and buffer salts. The coated MAP was then immediately foil sealed into the product pack, removed from the cleanroom and stored at 2 to 8° C. To simplify the manufacturing and testing of product three patches were applied to deliver the required doses. Subjects in the 15 μg groups received 3×5 μg patches; subjects in the 10 μg group received 2×5 μg and 1× placebo patches; subjects in 5 μg group received 1×5 μg and 2× placebo patches; subjects in the 2.5 μg group received 1×2.5 μg and 2× placebo patches; and subjects in the Om group received 3× placebo patches. The order of application was randomised and blinded.

The MAP applicator device (CAPD) was a hand-held spring powered device designed to reliably and reproducibly apply the MAP to the skin. The MAP applicator uses a mechanical force, generated by a spring, to accelerate the MAP to a sufficiently high velocity of 20 m/s over a short distance (<5 mm) for the dense array of micro-projections to breach the skin. The CAPD uses a magnet to attach, position and retain the MAP. The CAPD is a single use device and is used in conjunction with a skin conditioning ring. The skin conditioning ring contacts the skin around the area of MAP administration. Approximately 30 Newtons of down force is required to actuate the skin ring, resulting in a pre-conditioning of the skin for MAP administration.

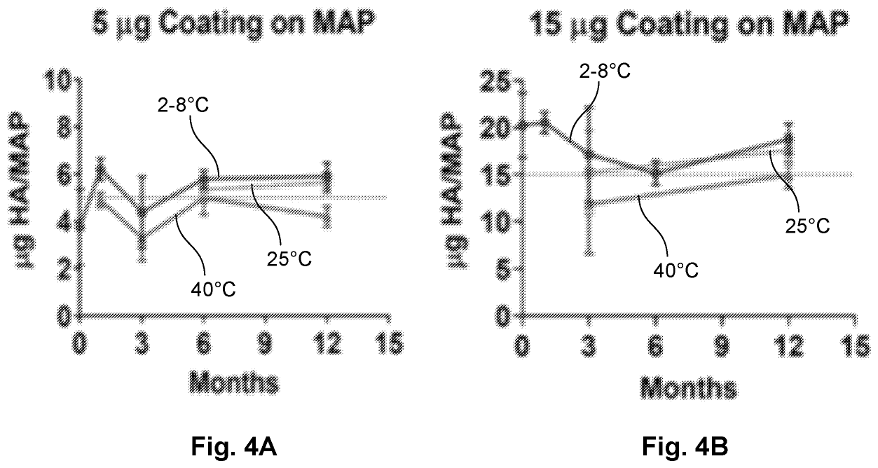

The A/Singapore/GP1908/2015 HA antigen dry-coated onto MAPs was shown to be stable (for 12 months up to a temperature of 40° C. following coating onto MAPs as measured by enzyme-linked immunoassay (Ref) (Fig Y). Placebo MAPs (for MAP-placebo/FA group) were gamma-sterilised (≥25 kGy, Steritech, Australia). All MAPs were placed into aluminium MediCan containers (Amcor, UK), foil-sealed, and stored at 2-8° C. before use (FIG. 4).

### Example 4

### Immunogenicity Evaluation

Serum samples were collected on days 1(pre), 4, 8, 22, and bland tested in hemagglutination inhibition (HAI) and virus microneutralisation (VMN) assays for days 1 and 22. HAI assays were performed as described previously (Fernando et al 2018). Briefly, Serum samples for HAI were treated with receptor destroying enzyme (Denka Seiken Co Ltd, Japan) and adsorbed to washed, packed turkey red blood cells (TRBC) for 30 min at room temperature (RT). TRBC were diluted to 1% v/v in PBS prior to testing. Two-fold serum dilutions starting from 1:10 were prepared and 4 HA Units/25 μL of A/Singapore/GP1908/2015 virus (WHO Collaborating Centre, Australia) were added to each test well and incubated for 45 min at room temperature (RT). TRBC were added and incubated for a further 30 minutes at RT. The HAI titre was the reciprocal of the highest dilution of the sera that completely inhibited agglutination of TRBC by the virus.

VMN assays were conducted according as described previously (Fernando et al 2018). Briefly, serum samples were heat inactivated for 56° C. for 30 min. Two-fold serum dilutions starting from 1:100 were prepared and 100 TCID₅₀ of A/Singapore/GP1908/2015 virus (WHO Collaborating Centre, Australia) were added to each test well. Prevention of infection of MDCK cells by A/Singapore/GP1908/2015 virus was tested using ELISA detection of influenza nucleoprotein.

Antibodies capable of mediating antibody-dependent cellular cytotoxicity (ADCC) were assayed using an ELISA that detected the ability of immobilized A/Sing MPH-specific antibodies to cross-link soluble recombinant FcγRIIIA receptor dimers (22). Serum samples collected on days 1 and 22 from subjects in groups MAP-FA-0, MAP-FA-15, MAP-UA-15 and IM-QIV-15 were tested. Briefly, 96 well Nunc Maxisorp plates (Thermofisher Scientific, USA) were coated for 16 hours at 4° C. with 50 ng of A/Singapore/GP1908/2015 HA in PBS, washed with PBS+0.05% Tween20 (PBST), and blocked with SuperBlock (Thermofisher Scientific), before addition of duplicate serially diluted serum samples (1:20-1:43,740). Plates were incubated at 37° C. for 1 hour then washed using PBST. An FcγRIIIA Val158 ectodomain biotin dimer (0.1 μg/mL) was added and incubated at 37° C. for 1 hour then washed using PBST. Antibody-FcγRIIIA complexes were detected using a 1:10,000 dilution of streptavidin-HRP (Thermofisher Scientific) and development with 3,3′,5,5′-tetramethylbenzidine substrate (Sigma-Aldrich, USA). The reaction was stopped with 0.16M H2SO4 and absorbance measured at 450 nm. Serum concentrations giving half-maximal signal (EC50) were determined using a fitted curve (4 parameter log regression) and GraphPad Prism (GraphPad Software, USA.

### Example 5

### Salivary IgA

Saliva samples were collected from subjects in the MAP-FA-0, MAP-FA-15, MAP-UA-15 and IM-QIV-15 groups on days 1, 4, 8 and 22. Subjects chewed on the cotton swab of a Salivette® saliva collector (Sarstedt, France) for approximately 1 minute. Following centrifugation, the supernatant (saliva) was stored at −80° C. Influenza specific IgA was detected by ELISA. Specifically, saliva samples serially diluted in 4 mg/mL BSA in PBS (PBSA) were added to Nunc Maxisorp plates (Thermofisher Scientific, USA) previously coated overnight with A/Singapore/GP1908/2015 HA MPH (60 μl per well at 2 μg/ml) and blocked with PBSA. The presence of A/Sing HA specific IgA was detected using HRP-1 conjugated goat anti-human polyclonal IgA (PA1-74395, Thermofisher Scientific, USA) and ABTS 232 substrate (Sera-Care, USA). The reaction was stopped with 1% SDS and absorbance measured at 405 nm.

### Example 6

### Memory B Cells

Peripheral blood mononuclear cells (PBMC) were collected and cryopreserved from subjects in the MAP-FA-0, MAP-FA-15, MAP-UA-15 and IM-QIV-15 groups on days 1 and 22 and stored in liquid 237 nitrogen until use. Recombinant HA proteins for use as flow cytometry probes for A/Michigan/45/2015, A/New Caledonia/20/1999 and the stabilised H1N1 stem domain were derived as previously reported (23). HA-specific B cells were identified within cryopreserved human PBMC by co-staining with HA probes conjugated to SA-PE, SA-APC, or SA-Ax488 (Thermofisher Scientific). Monoclonal antibodies for surface staining included: CD19-ECD (J3-119) (Beckman Coulter, USA), IgM-BUV395 (G20-127), CD21-BUV737 (B-ly4), IgD-Cy7PE (IA6-2), IgG-BV786 (G18-145) (BD 243 Biosciences, USA), CD14-BV510 (M5E2), CD3-BV510 (OKT3), CD8a-BV510 (RPA-T8), CD16-BV510 (3G8), CD10-BV510 (HI10a), CD27-BV605 (Biolegend, USA) and IgA-Vio450 (REA1014) 245 (Miltenyi, USA). Background B cells interacting with SA were excluded by staining with SA-BV510 (BD 246 Biosciences). Cell viability was assessed using Aqua Live/Dead amine-reactive dye (Thermofisher Scientific). Samples were collected using a BD Fortessa configured to detect 18 fluorochromes and analysis was performed using FlowJo software version 9.5.2 (TreeStar, USA).

### Example 7

### Flow-Cytometry of T Cells

Cytokine production by CD4+ and CD8+ T cells was assessed using a modification of the method described by Landry et al (24). PBMC were thawed, plated out at 1.5×106 per well and rested for 6 hours. After washing the PBMC were stimulated with either A/Sing MPH for 20 hours (20 μg/ml) or for 6 hours with a pool of overlapping synthetic peptides (17 amino acids long overlapping by 11 amino acids, 5 μg/ml) spanning the A/Sing HA sequence (Mimotopes Pty Ltd, Australia). Media only, and PMA/ionomycin were used as negative and positive controls respectively. Golgi blockers (monensin and brefeldin A) were added 5 hours before the end of incubation. Cells were labelled with surface stains Live/Dead Aqua (for viability), anti-CD3 BV785, anti-CD4 FITC, anti CD8 APC/Cy7 (all from Biolegend), and then fixed, permeabilised and labelled with anti IFN-γ Ax647, anti-TNF-α BV421, and anti-IL-2 PE (all from Biolegend). Samples were analysed on a Becton Dickinson LSR Fortessa X20 within 24 hours of the last wash step. Approximately 500,000 events were acquired, and the raw data were analysed initially using FlowJo (to obtain percentage positive values for each cytokine) before using SPICE (http://exon.niaid.nih.gov/spice) software to analyse background-subtracted values.

### Example 8

### Thermostability

A/Sing-coated HD-MAPs were stored at 2-8° C., 25° C.±2° C./60%±5% relative humidity (RH) and 40° C.±2° C./60%±5% RH for 12 months. At the designated timepoints, the coating was eluted from the HD-MAPs in 1 mL elution buffer (0.041% w/w Hypromellose, 0.0295% w/w trehalose dihydrate) using water bath sonication at 20-28° C., and the potency of HA determined by enzyme immunoassay (Bodle et al, 2013).

### Example 9

### Statistical Analysis

The fold increase in HAI titres and MN titres were compared between groups using a Student's test. The proportions of subjects seroprotected or seroconverted were compared between groups using a Pearson's chi-square test with continuity correction (SAS version 9.4, SAS Institute Inc., USA). As this was an early phase study, the exploratory efficacy analyses were not adjusted for the ADCC and memory B cell response assay, all groups were compared using Kruskal Wallis non-parametric tests (with no correction for multiple comparisons) and Dunn's multiple comparison post-tests. Within-group comparisons of cytokine production by CD4⁺ cells at day 1 and day 22 were made using the Wilcoxon rank sum test.

### Results

### Stability

To obtain stability data ahead of clinical manufacture a GLP stability study was performed testing 5 μg and 15 μg HA A/Sing loading on HD-MAPs. This loading range was selected to bracket the range of HA A/Sing loadings intended for use in the study. The A/Sing antigen coated at 5 μg and 15 μg per HD-MAP was stable when stored at 2-8° C. or 40° C. for at least 12 months (FIG. 4)

### Serological Response

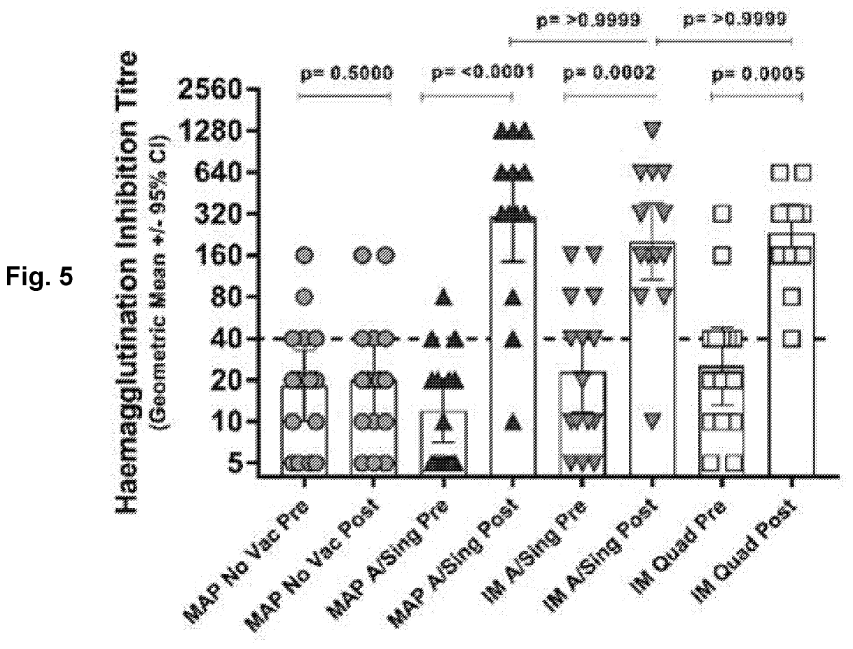

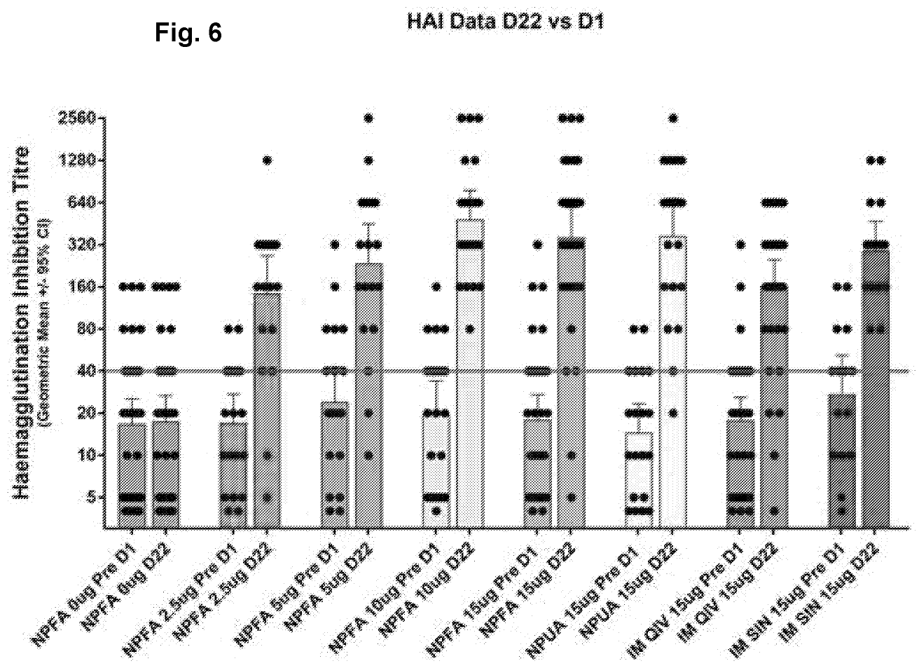

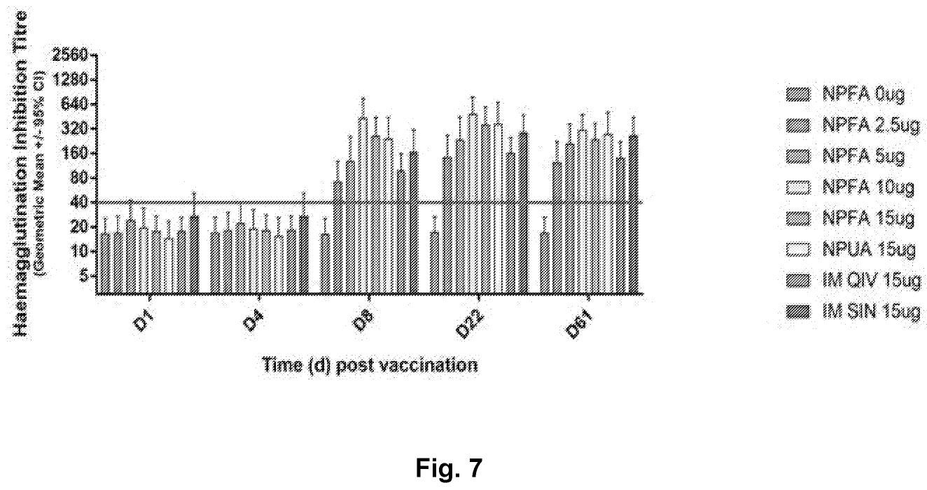

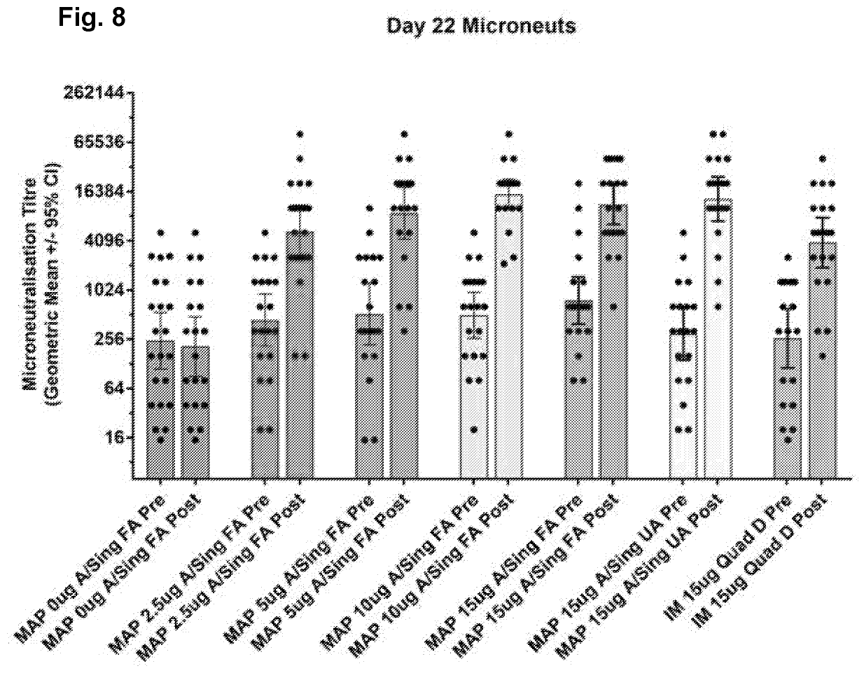

The geometric mean titres (GMTs) of HAI antibodies at days 1, 4, 8, 22 and 61 for subjects in part A are shown in FIG. 5. The data above demonstrate that 2.5 μg delivered to skin by the MAP induce an HAI titre that is not statistically different from that induced by either 15 μg of A/Singapore HA delivered using needle and syringe by itself or as a part of the quadrivalent vaccine. There is a 6 fold dose reduction of antigen when MAPs are used when compared to the 15 μg dose delivered intramuscularly in humans. It is possible that the dose could be reduced below 2.5 μg and still maintain antibody titers that are comparable to intramuscular injection. Although dose reductions have been shown in the mouse model for MAPs, this is the first time it is shown in humans. No statistical significant differences between the vaccinated groups were seen at day 22 time point. FIG. 6 is a plot of hemagglutinin inhibition titer for day 1 versus day 22 for several vaccine formulations in study A. FIG. 7 is a plot of hemagglutinin inhibition titer versus time for study A. FIG. 8 is a plot of microneutralization titer at day 22 for study A.

The HAI GMTs, seroprotection and seroconversion rates and fold-increase in GMT titres are shown in Table 1, which show Hemagglutination inhibition responses to vaccination in terms of seroprotection, seroconversion and fold-increase in GMT above pre-vaccination levels for part B.

TABLE 1

| | | MAP- | MAP- | MAP- | MAP- | MAP- | MAP- | IV- |
|---|---|---|---|---|---|---|---|---|
| Day | | FA-15 | FA-10 | FA-5 | FA-2.5 | FA-0 | UA-15 | QIV-15 |
| Day 1 | GMT (95% CI) | 20.7 | 19.3 | 23.0 | 16.2 | 13.2 | 13.2 | 12.3 |
| | | (11.7, 36.7) | (11.0, 33.9) | (12.5, 42.3) | (9.8, 26.9) | (6.7, 26.0) | (7.7, 22.7) | (7.2, 21.1) |
| | Seroprotection | 10/20 | 9/20 | 8/20 | 8/20 | 6/20 | 6/20 | 7/20 |
| | N (%) | (50%) | (45%) | (40%) | (40%) | (30%) | (30%) | (35%) |
| Day 4 | GMT (95% CI) | 20.7 | 18.7 | 23.1 | 17.4 | 13.2 | 14.1 | 12.7 |
| | | (11.4, 37.7) | (10.8, 32.2) | (12.3, 43.7) | (10.2, 29.7) | (6.7, 26.0) | (8.0, 25.0) | (7.3, 22.3) |
| | Seroprotection | 10/20 | 9/20 | 7/19 | 8/20 | 6/20 | 7/20 | 7/20 |
| | N (%) | (50%) | (45%) | (37%) | (40%) | (30%) | (35%) | (35%) |
| | Serocoversion | 0/20 | 0/20 | 0/19 | 0/20 | 0/20 | 0/20 | 0/20 |
| | N (%) | (0%) | (0%) | (0%) | (0%) | (0%) | (0%) | (0%) |
| | GMT fold | 1.0 | 1.0 | 1.0 | 1.0 | 1.0 | 1.0 | 1.0 |
| | increase 95% CI | (0.9, 1.1) | (0.9, 1.0) | (1.0, 1.1) | (1.0, 1.2) | (1.0, 1.0) | (1.0, 1.2) | (1.0, 1.1) |
| Day 8 | GMT (95% CI) | 218.6 | 437.1 | 125.5 | 72.1 | 13.7 | 242.5 | 82.8, |
| | | (111.9, 427.0)* | (254.3, 751.3)** | (61.4, 256.9) | (40.4, 128.7) | (6.8, 27.6) | (133.2, 441.5)* | (42.4, 161.8) |
| | Seroprotection | 19/20 | 20/20 | 17/20 | 18/20 | 6/20 | 18/20 | 17/20 |
| | N (%) | (95%) | (100%) | (85%) | (90%) | (30%) | (90%) | (85%) |
| | Serocoversion | 17/20 | 18/20 | 12/20 | 11/20 | 0/20 | 18/20 | 15/20 |
| | N (%) | (85%) | (90%) | (60%) | (55%) | (0%) | (90%) | (75%) |
| | GMT fold | 10.6 | 22.6 | 5.5 | 4.4 | 1.0 | 18.4 | 6.7 |
| | increase 95% CI | (4.8, 23.2) | (10.9, 47.1)** | (3.0, 10.0) | (2.8, 7.0) | (1.0, 1.1) | (10.3, 32.9)** | (4.1, 11.1) |
| Day 22 | GMT (95% CI) | 320.0 | 485.0 | 234.3 | 144.2 | 13.2 | 367.6 | 139.3 |
| | | (160.5, 638.1) | (301.5, 780.2) | (121.9, 450.0) | (77.9, 267.0) | (6.7, 26.0) | (197.9, 682.7)* | (79.3, 244.5) |
| | Seroprotection | 19/20 | 20/20 | 18/20 | 18/20 | 6/20 | 19/20 | 17/20 |
| | N (%) | (95%) | (100%) | (90%) | (90%) | (30%) | (95%) | (85%) |
| | Serocoversion | 17/20 | 18/20 | 14/20 | 16/20 | 0/20 | 18/20 | 15/20 |
| | N (%) | (85%) | (90%) | (70%) | (80%) | (0%) | (90%) | (75%) |
| | GMT fold | 15.5 | 25.1 | 10.2 | 8.9 | 1.0 | 27.9 | 11.3 |
| | increase 95% CI | (6.7, 35.7) | (13.4, 46.9)* | (5.1, 20.4) | (5.0, 15.8) | (1.0, 1.0) | (15.0, 51.7)* | (6.8, 18.8) |
| Day 61 | GMT (95% CI) | 211.1 | 309.1 | 211.1 | 125.5 | 12.7 | 278.6 | 109.3 |
| | | (121.7, 366.3) | (199.1, 479.9)** | (121.7, 366.3) | (71.0, 221.9) | (6.4, 25.5) | (152.7, 508.1)* | (59.4, 200.9) |
| | Seroprotection | 19/20 | 20/20 | 19/20 | 18/20 | 6/20 | 19/20 | 17/20 |
| | N (%) | (95%) | (100%) | (95%) | (90%) | (30%) | (95%) | (85%) |
| | Serocoversion | 176/20 | 18/20 | 14/20 | 15/20 | 0/20 | 18/20 | 13/20 |
| | N (%) | (80%) | (90%) | (70%) | (75%) | (0%) | (90%) | (65%) |
| | GMT fold | 10.2 | 16.0 | 9.2 | 7.7 | 1.0 | 21.1 | 8.9 |
| | increase 95% CI | (5.1, 20.5) | (9.6, 26.8) | (4.9, 17.4) | (4.4, 13.6) | (0.9, 1.0) | (12.0, 37.0)* | (5.1, 15.4) |

*indicates p 442 < 0.05 and
**indicates p < 0.01 compared to the IM-QIV-15 group by Student's t-test (fold increase and GMTs) and using a Pearson's chi-square test with 443 continuity correction for proportion of subjects seroconverted or seroprotected.

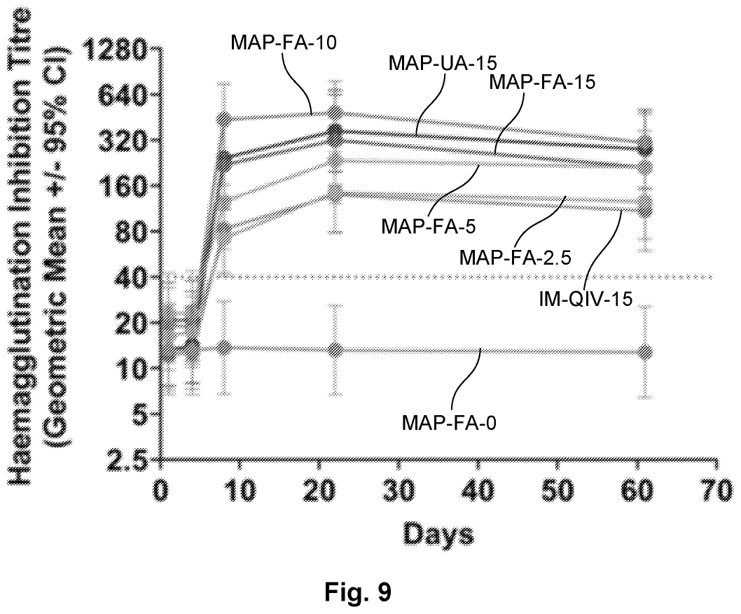

The geometric mean titres (GMTs) of HAI antibodies at days 1, 4, 8, 22 and 61 for subjects in part B are shown in FIG. 9. There was no increase in HAI titre in subjects receiving uncoated HD-MAPs. In subjects receiving vaccine, either by HD-MAP or IM, titres did not increase above baseline at day 4, but at day 8 the GMTs were highest in the MAP-FA-10 (GMT 437, 254-751 95% CI), MAP-UA-15 (GMT 243, 133-442 95% CI) and MAP-FA-15 (GMT 219, 112-427 95% CI) groups. The increases in GMT titres from baseline were significantly higher at day 8 (MAP-FA-10 p=0.0002, MAP-UA-15 p=0.0167, MAP-FA-15 p=0.0384) than in the IM-QIV-15 group (GMT 83, 42-161 95% CI). Titres continued to increase in all active groups until day 22, and remained significantly higher than the IM-QIV-15 group in the MAP-FA-10 and MAP-UA-15 groups at day 22 (p=0.0011 and p=0.0201 respectively), and in the MAP-FA-10 and MAP-UA-15 groups at day 61 (p=0.0062 and p=0.0277 respectively). The GMT in the MAP-FA-2.5 group (subjects that received ⅙ the standard dose of HA), was not significantly different from the GMT in the IM-QIV-15 group at any time point (day 4 p=0.4034; day 8 p=0.7449; day 22 p=0.9312, day 61 p=0.7297). Furthermore, at day 22 the HAI 405 GMTs were similar in the MAP-FA-15 (GMT 320, 161-638 95% CI) and MAP-UA-15 (GMT 368, 198-683 95% CI) groups, indicating that the site of HD-MAP application did not affect the subsequent antibody response.

The 429 fold-increases in GMT at day 8 were significantly higher in the MAP-FA-10 and MAP-UA-15 groups 430 (22.6 and 18.4 respectively), compared with the IM-QIV-15 group (1.0) (p=0.0069 and p=0.0095 431 respectively) indicating a more rapid antibody response compared with IM injection. The fold-432 changes from baseline remained significantly higher in the MAP-UA-15 group at days 22 and days 61 433 (p=0.0240 and p=0.0265 respectively). The HAI titers observed in part A subjects receiving vaccine delivered by HD-MAP were not significantly different from the corresponding group in part B at any time-point (i.e. A-MAP-FA-15 compared with MAP-FA-15, all p values>0.4180) indicating consistency of delivery and induction of antibody. The GMTs induced by IM-QIV-15 in parts A and B were also not significantly different at days 4, 8, and 22 but were higher in part A at day 61 (p=0.0320).

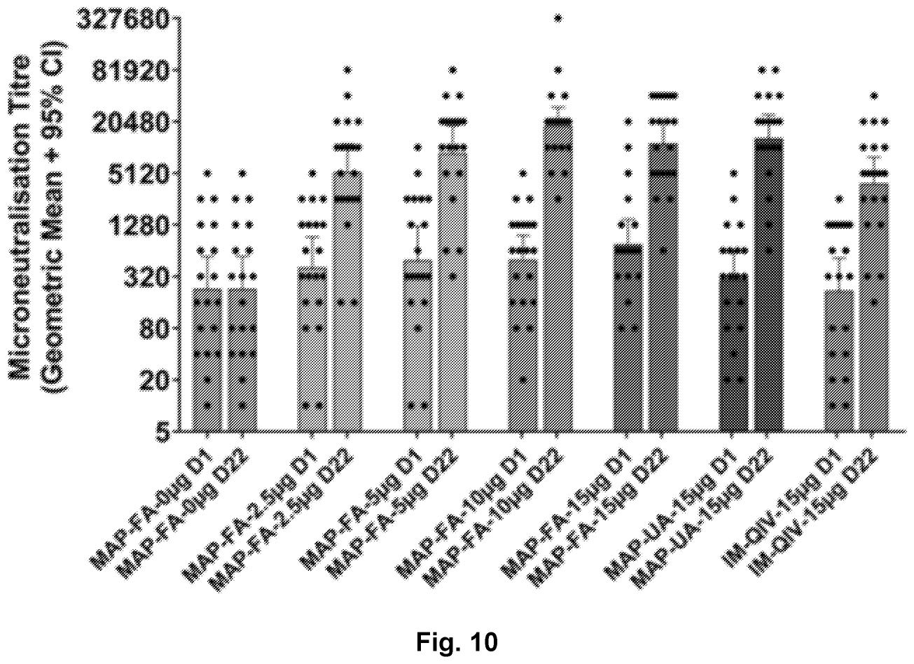

MN assays were carried out on serum samples from all part B subjects at days 1 (pre-vaccination) and day 22. As with the HAI antibodies, there was an increase in titre from day 1 to day 22 in all treatment groups that received the vaccine (FIG. 10). The MN titres at day 22 in the MAP-FA-10 and MAP-UA-15 groups were significantly higher than the IM-QIV-15 group (p=0.0005 and p=0.0096 respectively). As with the HAI results, the MN GMTs at day 22 in the MAP-FA-2.5 (⅙th dose) (GMT 5,301, 2,509-11,196 95% CI) and IM-QIV-15 (GMT 3,880 1,924-7,824 95% CI) groups were similar (FIG. 10).

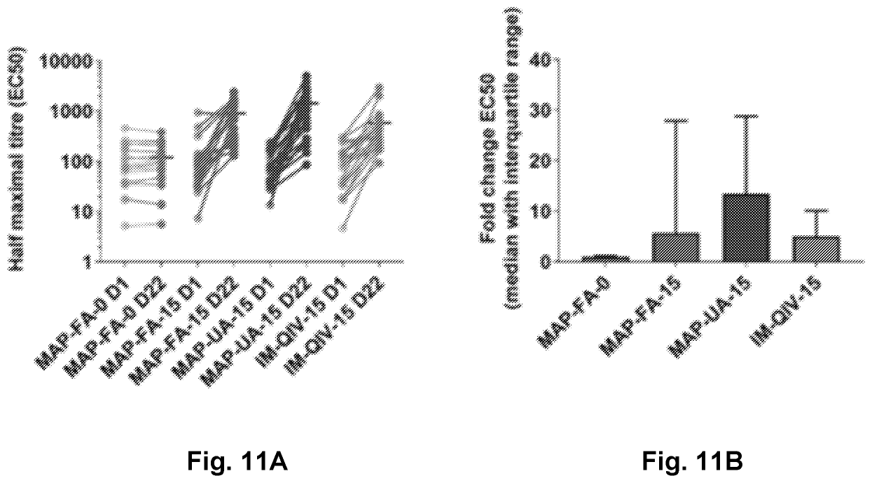

Titres of HA-specific FcR-binding antibodies capable of mediating ADCC, were assayed at days 1 and 22. The mid-point titres increased significantly following vaccine delivery in the MAP-FA-15, MAP-FA-0, MAP-UA-15 and IM-QIV-15 groups (p<0.001, p<0.001, p=0.002 respectively) but not in the MAP-FA-0 group (p>0.99) (Figure X3A). There was no significant difference between the mid-point titres at day 22 in these three active groups (p>0.99 for all comparisons), nor was there a difference when the results were expressed as fold-change from baseline, due the degree of intra-group variation (FIG. 11).

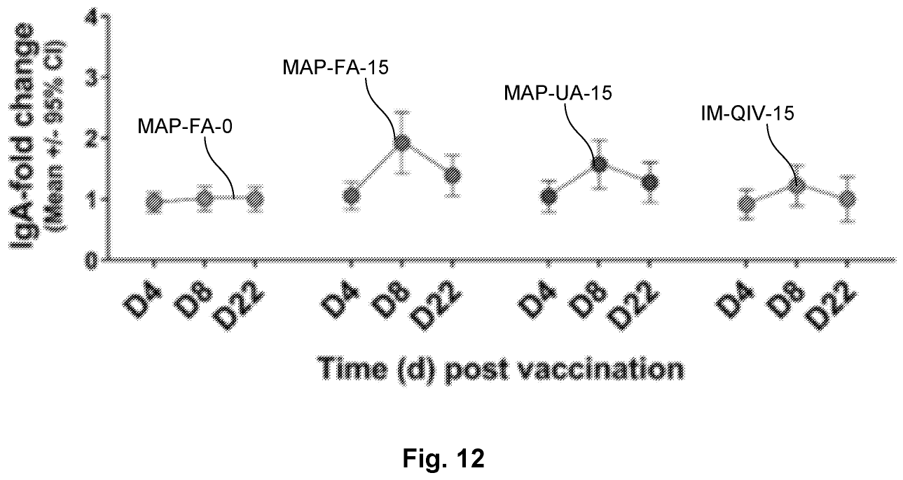

Influenza-specific IgA in saliva was assayed by ELISA in samples taken at days 1, 4, 8 and 22 from subjects in groups MAP-FA-0, MAP-FA-15, MAP-UA-15 and IM-QIV-15. There was no significant increase in titre compared with day 1 in any of the groups. There was however, a 1.92 and 1.57-fold increase over baseline in the MAP-FA-15 and MAP-UA-15 groups at day 8 compared with no increase for the MAP-FA-0 (1.01-fold) and 1.22-fold increase in the IM-QIV-15 groups at the same timepoint. IgA titres had returned to near-baseline levels at day 22. (FIG. 12)

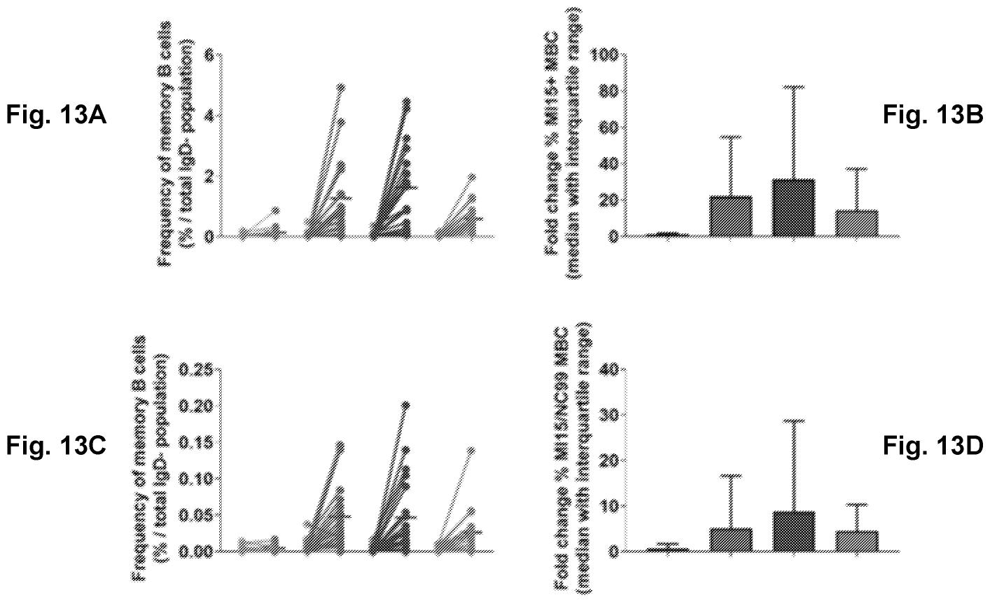

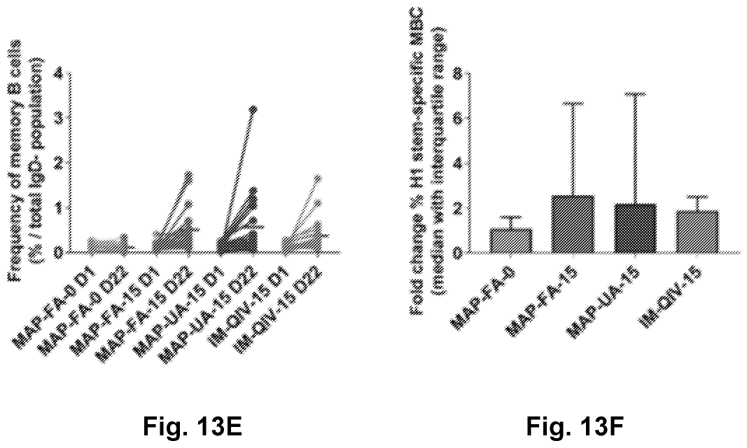

A flow cytometry-based assay using fluorescently-labeled recombinant HA was used to assess frequency and specificity of HA-specific B cells following immunization. Frequencies of memory B cells (MBC) binding a HA-Michigan probe (antigenically matched to A/Singapore/GP1908/2015 were elevated at day 22 following immunization with either QIV or the active HD-MAPs (MAP-FA-15 p<0.0001, MAP-UA-15 p<0.0001 and IM-QIV-15 p<0.0001, but not the placebo group (MAP-FA-0 p<0.0001). The frequencies of the HA-Michigan-specific MBC at day 22 were not significantly different in the three vaccine groups however (p>0.99 for all comparisons. Using binding to A/New Caledonia/99 probe to assess H1N1 cross-reactivity, only a small portion of the A/Michigan/15-binding cells displayed cross-reactivity recognition of HA. There was a significant increase in frequency between day 1 and day 22 in the MAP-FA-15 (p<0.0001) and MAP-UA-15 (p<0.0001) groups and to a lesser extent in the IM-QIV-15 group (p=0.0522). Again there was no difference in the MBC frequencies in the vaccine groups at day 22 (p<0.99 for all comparisons). A similar pattern was seen with cross-reactive B cells binding HA-stalk domain probe. There was a significant expansion from day 1 to day 22 in the MAP-FA-15 (p=0.006) and IM QIV-15 (p=0.468) groups. The expansion in the MAP-UA-15 groups did not achieve significance, probably due to intragroup variability (p=0.167). There was no difference in the stalk-reactive MBC frequencies between active HD-MAP and IM groups at day 22 (p>0.99 for all comparisons). FIG. 13A-F

T cell responses were assessed by analysing the frequencies of influenza-specific CD4+ and CD8+ T producing IFN-γ, IL-2 and TNF-α in PBMC harvested on days 1 and 22 from subjects in groups MAP-FA-0, MAP-FA-15, MAP-UA-15, and IM-QIV-15. PBMC were stimulated with either A/Sing MPH or 536 overlapping peptides spanning the A/Sing HA sequence.

There was an increase in the overall frequency of CD4+ cells producing IFN-γ, IL-2 or TNF-α following in vitro stimulation with the peptides at day 22 compared to day 1 in the MAP-FA-15, MAP-UA-15 and IM-QIV-15 groups but not the MAP-FA-0 group (FIG. 8A). In particular, there was a significant increase in the abundance of polyfunctional CD4+ T-cells expressing all three cytokines (IFN-γ, TNF-α and IL-2), at day 22 compared with day 1 for the MAP-FA-15 (p=0.0002), MAP-UA-15 (p=0.0045), and IM-QIV-15 (p=0.0110) groups. There was also a significant increase in CD4+ cells producing TNF-α and IL-2 in the MAP-FA-15 (p=0.0008), MAP-UA-15 (p=0.0003) and IM-QIV (p=0.0012) groups. The MAP-FA-15 group also showed a significant increase in CD4+ cells expressing TNF-α alone (p=0.0053), and TNF-α with IFN-γ (p=0.0013) (FIG. 8A). There were no statistically significant differences in the proportions of CD4+ T cells producing any of the cytokine combinations at day 22 when the MAP-FA-15, MAP-UA-15 and IM-QIV-15 groups were compared with one another (p>0.0565 for all comparisons).

The overall frequency of cytokine-producing CD4+ cells pre and post-vaccination was greater following stimulation with A/Sing MPH compared with the overlapping peptides, presumably due to the greater number of epitopes present within the MPH preparation. A/Sing MPH stimulation appeared to induce more CD4+ cells producing TNF-α alone compared with peptide stimulation. There were not however, any statistically significant differences in the proportions of CD4+ T cells producing each of the cytokine combinations at day 22 when the MAP-FA-15, MAP-UA-15 and IM-QIV-15 groups were compared with one another (p>0.0515 for all comparisons.

Day 1 and day 22 CD8+ T cell responses to the peptide pools and A/Sing MPH were also measured but were weak in comparison with the CD4+ responses. The weak CD8+ T cell responses were not surprising considering that the nature of antigen used for re-stimulation (inactivated, split A/Sing MPH and 17 amino-acid peptides) favored stimulation and detection of CD4+ T cells.

This study demonstrated MAP dose sparing in the clinic. The safety and reactogenicity profiles of the HD-MAPs were very similar to those observed with the silicon Nanopatches using a similar H1N1 antigen, A/California/7/2009 (3,18), and the fact that erythema is still present seven days after vaccination is also consistent with intradermal (ID) delivery of influenza vaccines. No differences in the HAI or MNT responses were found following HD-MAP application to the upper arm or volar surface of the forearm.

In terms of the proportion of subjects seroprotected and seroconverting, the HAI responses induced by HD-MAP delivery in this study were similar to those seen previously with needle and syringe ID injection of inactivated influenza vaccine (IIVs). However, the more rapid antibody response seen with HD-MAP delivery as indicated by higher HAI titres at the early day 8 time-point have not been seen with ID injection of IIVs unless the ID injection site was pre-treated with the topical adjuvant Imiquimod, a TLR7 agonist (34). Achieving higher titres, sooner after vaccination by using the HD-MAP to deliver seasonal influenza or travel vaccines would be beneficial for vaccine recipients and would be particularly valuable if it was shown to apply to vaccines against pandemic influenza strains and vaccines used in outbreak response.

Influenza vaccines that induce more broadly protective and longer lasting immunity than current seasonal vaccines are needed to limit the consequences of epidemic and pandemic influenza. Studies have suggested that ADCC-mediated antibodies recognize epitopes that are more conserved than those bound by neutralizing antibodies and might contribute to protection against heterologous strains. The induction of antibodies with ADCC-induced potential followed a similar pattern of response to HAI and MN data with slightly higher titers being observed in groups vaccinated with the HD-MAP compared to IM injection. The frequencies of B cells recognizing HA-stalk and an historic H1N1 HA probe also increased to a similar extent following IM of HD-MAP vaccination.

Several HD-MAP groups showed statistically higher responses at days 8, 22 and 61 after vaccination compared with IM injection, and the ⅙th dose induced antibody levels that were equivalent to the full-dose IM.

Synthetic polymer MAP vaccination is safe and acceptable to the subjects. MAPs can deliver split inactivated A/Singapore/GP1908/2015 (H1N1) virus antigen (2.5 μg HA) to human skin, an HA specific antibody response equivalent to that generated by the conventional 15.0 μg HA IM vaccination could be achieved. This 6-fold dose reductions means the cost of vaccines could be reduced and more vaccines could be made available especially when the availability of antigen is a limiting factor as in the case of pandemics. Furthermore, reduction of costs of expensive vaccines such as anti-cervical cancer vaccine, will help resource-poor countries to access these expensive vaccines readily, provided these dose reductions are possible for majority of other vaccines in addition to influenza vaccine.

MAPs delivery of doses as low as 2.5 μg HA induced similar HAI and MNT titers to IM QIV (15 μg HA/dose). Seroconversion and seroprotection rates at day 8 are higher with MAPs delivery. No difference seen between forearm and upper arm application of MAPs. MAPs have excellent high-temperature stability.

Throughout this specification and claims which follow, unless the context requires otherwise, the word “comprise”, and variations such as “comprises” or “comprising”, will be understood to imply the inclusion of a stated integer or group of integers or steps but not the exclusion of any other integer or group of integers. As used herein and unless otherwise stated, the term “approximately” means ±20%.

Persons skilled in the art will appreciate that numerous variations and modifications will become apparent. All such variations and modifications which become apparent to persons skilled in the art, should be considered to fall within the spirit and scope that the invention broadly appearing before described.

1. A method of stimulating an immune response in a human, comprising the step of administering to the human a vaccine dose which is coated onto a microprojection array patch (MAP).

2. The method of claim 1, wherein the MAP comprises a base and a number of solid, non-porous projections extending from the base made of synthetic polymer, wherein at least one projection comprises an uncoated support section which transitions into end section which is dry-coated with vaccine.

3. The method of claim 1 or claim 2, wherein the projections are about 200 to 300 μm in length and about 100 to about 120 μm in width at the base and the density of the projections is from about 1000 to about 5000 projections/cm² and the MAP weighs between 0.1 to 0.6 grams.

4. The method of claim 3, wherein the MAP is made of a synthetic polymer.

5. The method of claim 4, wherein the synthetic polymer is a liquid crystal polymer.

6. The method of any one of the claims 1 to 5, wherein administration of the composition to the human provides protective immunity against an infection consequent to exposure of the human to a source of antigen.

7. The method of any one of the claims 1 to 6, wherein the human is from 49 to 64 years old.

8. The method of any one of the claims 1 to 6, wherein the human is at least 65 years old.

9. The method of any one of the claims 1 to 8, wherein the dose is at least one dose selected from the group consisting of a 0.5 μg dose, 1 μg dose, 2 μg dose, 2.5 μg dose, 3 μg dose, 4 μg dose, 5 μg dose, 6 μg dose, 7 μg dose, 8 μg dose, 10 μg dose, 15 μg dose, 20 μg dose, 25 μg dose and a 30 μg dose.

10. The method of claim 9, wherein the dose is at least one dose selected from the group consisting of a 2.5 μg dose, 5 μg dose, 10 μg dose and a 15 μg dose.

11. The method of any one of the claims 1 to 10, wherein the vaccine dose comprises one or more influenza antigens.

12. The method of claim 11, wherein the influenza antigen is a hemagglutinin influenza antigen

13. The method of claim 12, wherein the influenza antigen is an influenza A antigen.

14. The method of claim 12, wherein the influenza antigen is an influenza B antigen.

15. The method of claim 12, wherein the influenza antigen is an influenza C antigen.

16. The method of any one of the claims 1 to 15, further including the step of administering at least one subsequent dose of the vaccine to the human.

17. A method of stimulating an immune response in a human population, comprising the step of administering to the human population vaccine doses which are dry-coated onto a microprojection array patch (MAP) and inserted into the skin of the humans in the population, wherein the seroconversion rate in the human population is at least 85% as measured at least 8 days after the administration of the vaccine.

18. A method of stimulating an immune response in a human population, comprising the step of administering to the human population vaccine doses which are dry-coated onto a microprojection array patch (MAP) and inserted into the skin of the humans in the population, wherein the seroprotection rate in the human population is at least 95% as measured at least 8 days after the administration of the vaccine.

19. The method of claim 17 or 18, wherein the vaccine dose comprises one or more influenza antigens.

20. The method of claim 19, wherein the influenza antigen is a hemagglutinin influenza antigen.

21. The method of claim 20, wherein the influenza antigen is an influenza A antigen.

22. The method of claim 20, wherein the influenza antigen is an influenza B antigen.

23. The method of claim 20, wherein the influenza antigen is an influenza C antigen.

24. The method of claim 20 wherein the dose comprises between 2.5 to 15 μg hemagglutinin influenza antigen.

25. A method of stimulating an immune response in a human population, comprising the step of administering to the human population vaccine doses which are dry-coated onto a microprojection array patch (MAP) and inserted into the skin of the humans in the population, wherein the geometric mean titres (GMT) in the human population is at least sixfold greater than the GMT compared to intramuscular injection of the same dose of vaccine as measured at least 8 days after the administration of the vaccine.

26. The method of claim 25 wherein the GMT in the human population is from about sixfold to about tenfold greater than the GMT compared to intramuscular injection of the same dose of vaccine as measured at least 8 days after the administration of the vaccine.

27. Apparatus for stimulating an immune response in a human, the apparatus comprising a vaccine dose which is coated onto a microprojection array patch (MAP).

28. The apparatus of claim 27, wherein the MAP comprises a base and a number of solid, non-porous projections extending from the base made of synthetic polymer, wherein at least one projection comprises an uncoated support section which transitions into end section which is dry-coated with vaccine.

29. The apparatus of claim 27 or claim 28, wherein the projections are about 200 to 300 μm in length and about 100 to about 120 μm in width at the base and the density of the projections is from about 1000 to about 5000 projections/cm² and the MAP weighs between 0.1 to 0.6 grams.

30. The apparatus of claim 29, wherein the MAP is made of a synthetic polymer.

31. The apparatus of claim 30, wherein the synthetic polymer is a liquid crystal polymer.

32. The apparatus of any one of the claims 27 to 31, wherein administration of the composition to the human provides protective immunity against an infection consequent to exposure of the human to a source of antigen.

33. The apparatus of any one of the claims 27 to 32, wherein the human is from 49 to 64 years old.

34. The apparatus of any one of the claims 27 to 32, wherein the human is at least 65 years old.

35. The apparatus of any one of the claims 27 to 34, wherein the dose is at least one dose selected from the group consisting of a 0.5 μg dose, 1 μg dose, 2 μg dose, 2.5 μg dose, 3 μg dose, 4 μg dose, 5 μg dose, 6 μg dose, 7 μg dose, 8 μg dose, 10 μg dose, 15 μg dose, 20 μg dose, 25 μg dose and a 30 μg dose.

36. The apparatus of claim 35, wherein the dose is at least one dose selected from the group consisting of a 2.5 μg dose, 5 μg dose, 10 μg dose and a 15 μg dose.

37. The apparatus of any one of the claims 27 to 36, wherein the vaccine dose comprises one or more influenza antigens.

38. The apparatus of claim 37, wherein the influenza antigen is a hemagglutinin influenza antigen

39. The apparatus of claim 38, wherein the influenza antigen is an influenza A antigen.

40. The apparatus of claim 38, wherein the influenza antigen is an influenza B antigen.

41. The apparatus of claim 38, wherein the influenza antigen is an influenza C antigen.

42. Apparatus for stimulating an immune response in a human population, the apparatus comprising vaccine doses which are dry-coated onto a microprojection array patch (MAP) configured to be inserted into the skin of humans in the population so that the seroconversion rate in the human population is at least 85% as measured at least 8 days after the administration of the vaccine.

43. Apparatus for stimulating an immune response in a human population, the apparatus comprising vaccine doses which are dry-coated onto a microprojection array patch (MAP) configured to be inserted into the skin of the humans in the population such that the seroprotection rate in the human population is at least 95% as measured at least 8 days after the administration of the vaccine.

44. The apparatus of claim 42 or 43, wherein the vaccine dose comprises one or more influenza antigens.

45. The apparatus of claim 44, wherein the influenza antigen is a hemagglutinin influenza antigen.

46. The apparatus of claim 45, wherein the influenza antigen is an influenza A antigen.

47. The apparatus of claim 45, wherein the influenza antigen is an influenza B antigen.

48. The apparatus of claim 45, wherein the influenza antigen is an influenza C antigen.

49. The apparatus of claim 45, wherein the dose comprises between 2.5 to 15 μg hemagglutinin influenza antigen.

50. Apparatus for stimulating an immune response in a human population, the apparatus comprising vaccine doses which are dry-coated onto a microprojection array patch (MAP) configured to be inserted into the skin of the humans in the population such that the GMT in the human population is at least sixfold greater than the GMT compared to intramuscular injection of the same dose of vaccine as measured at least 8 days after the administration of the vaccine.

51. The apparatus of claim 25 wherein the GMT in the human population is from about sixfold to about tenfold greater than the GMT compared to intramuscular injection of the same dose of vaccine as measured at least 8 days after the administration of the vaccine.
